(19) United States
(12) Patent Application Publication
(10) Pub. No.: US 2025/0263774 A1
(43) Pub. Date: Aug. 21, 2025

(54) DISCOVERING NEW XENOBIOTIC SUBSTRATES OF AN UDP-GLYCOSYLTRANSFERASE WITH A HIGH-THROUGHPUT METHOD

(71) Applicants: UNIVERSITY OF NORTH TEXAS, Dallas, TX (US); UNIVERSITY OF HOUSTON SYSTEM, Houston, TX (US); SANARENTERO LLC, Pearland, TX (US)

(72) Inventors: Clement T.Y. CHAN, Dallas, TX (US); Ming HU, Houston, TX (US); Rashim SINGH, Houston, TX (US); Dinh Bui, Houston, TX (US)

(21) Appl. No.: 19/060,083

(22) Filed: Feb. 21, 2025

Related U.S. Application Data
(60) Provisional application No. 63/556,230, filed on Feb. 21, 2024.

(51) Int. Cl. C12Q 1/48; G01N 21/64

(52) U.S. Cl. CPC C12Q 1/48; G01N 21/6428; G01N 2021/6439; G01N 2333/91102

(57) ABSTRACT

A method of rapidly determining a UGT-transferase substrate includes contacting a UGT-transferase with a UDP-sugar conjugate and a probe to form an assay mixture, subjecting the assay mixture to reaction conditions to form a reacted mixture, contacting the reacted mixture with a UDP-reactive dye under conditions suitable to associate the UDP with the UDP-reactive dye, and spectrophotometrically determining the amount of free UDP, wherein if the concentration of free UDP is greater than a concentration of free UDP in a reference sample prepared in the absence of the UGT-transferase, then the probe molecule is a designated a substrate.

### CROSS-REFERENCE TO RELATED APPLICATIONS

This application claims benefit of U.S. provisional patent application Ser. No. 63/556,230 filed Feb. 21, 2024, and entitled “DISCOVERING NEW XENOBIOTIC SUBSTRATES OF AN UDP-GLYCOSYLTRANSFERASE WITH A HIGH-THROUGHPUT METHOD,” which is hereby incorporated herein by reference in its entirety for all

### STATEMENT REGARDING FEDERALLY SPONSORED RESEARCH OR DEVELOPMENT

This invention was made with government support under contract nos. R35GM142421, R41CA261292, and R41CA275454 all awarded by The National Institutes of Health. The government has certain rights in the invention.

### TECHNICAL FIELD

The present disclosure relates generally to transferases. More particularly the present disclosure relates to novel methods for the identification of transferase substrates.

### BACKGROUND

Uridine diphosphate-glycosyltransferases (UGTs) are a superfamily of enzymes found in animals, plants, fungi, and germs that catalyze the covalent addition of sugars from nucleotide uridine diphosphate (UDP) sugar donors to functional groups on a variety of lipophilic compounds. There are 22 UGTs in humans. UGTs found in the endoplasmic reticulum membrane catalyzes the attachment of the hemiacetal hydrogen bond of glucuronic acid (UDPGA) to a range of compounds containing functional groups, such as hydroxy, carboxylic, amino, and sulfhydryl groups to create esters or glycosides. Glycosyltransferases are present in a broad range of organisms, including animals, plants, and microbes. These enzymes transfer glycosyl groups from activated sugars, such as uridine-5′-diphosphate-glucose (UDP-glucose), to target substrates. Substrates of glycosyltransferases are highly diverse, which can be alkaloids, antibiotics, plant hormones, nucleic acids, proteins, and toxic xenobiotics. As many of these substrates are important in medicinal and agricultural applications, researchers have put a huge effort in studying the protein superfamily of glycosyltransferases and exploring the use of these enzymes in metabolic engineering and drug metabolism. UGTs are known for accepting a broad variety of substrates and they derivatize xenobiotics for detoxification. However, most UGT homologs have not been well-characterized, and their potential for biomedical and environmental applications is underexplored.

One main challenge in studying glycosyltransferases is the time and labor required to characterize their substrate specificity. Conventionally, liquid chromatography approaches are used to isolate compounds after a glycosyltransferase reaction, such that presence of glycosylated products can be detected. It often requires modifying the chromatographic method to characterize different substrate candidates and the throughput of substrate screening is low; these limitations highly restrict the characterization of substrate profile for these enzymes. An ongoing need exists for rapid modalities of identifying substrates for these enzymes and potentially expanding their application.

### SUMMARY

In some embodiments, a method of rapidly determining a UGT-transferase substrate comprises contacting a UGT-transferase with a UDP-sugar conjugate and a probe to form an assay mixture, subjecting the assay mixture to reaction conditions to form a reacted mixture; contacting the reacted mixture with a UDP-reactive dye under conditions suitable to associate the UDP with the UDP-reactive dye, and spectrophotometrically determining the amount of free UDP, wherein if the concentration of free UDP is greater than a concentration of free UDP in a reference sample prepared in the absence of the UGT-transferase, then the probe molecule is a designated a substrate.

In some embodiments, a method of rapidly determining a UGT-transferase substrate comprises contacting a UGT-transferase with a UDP-glucose conjugate and a probe to form an assay mixture, subjecting the assay mixture to reaction conditions to form a reacted mixture, contacting the reacted mixture with a UDP-reactive dye under conditions suitable to associate the UDP with the UDP-reactive dye wherein the UDP-reactive dye is fluorescent, and spectrophotometrically determining the amount of free UDP.

Aspects described herein comprise a combination of features and characteristics intended to address various shortcomings associated with certain prior devices, systems, and methods. The foregoing has outlined rather broadly the features and technical characteristics of the disclosed aspects in order that the detailed description that follows may be better understood.

The various characteristics and features described above, as well as others, will be readily apparent to those skilled in the art upon reading the following detailed description, and by referring to the accompanying drawings. It should be appreciated that the conception and the specific aspects disclosed may be readily utilized as a basis for modifying or designing other structures for carrying out the same purposes as the disclosed aspects. It should also be realized that such equivalent constructions do not depart from the spirit and scope of the principles disclosed herein.

### BRIEF DESCRIPTION OF THE DRAWINGS

For a detailed description of various exemplary aspects, reference will now be made to the accompanying drawings in which:

FIG. 1A is a schematic of the method for detection of UDP-glycosyltransferase activities is based on the formation of a UDP-dye complex, which increases fluorescence of the sample.

FIG. 11B is a bar graph enzymatic activity of UGT71 G1 with 3-hydroflavone as the substrate. As a control experiment, the assay was also performed with the sample conditions except without the enzyme UGT71G1.

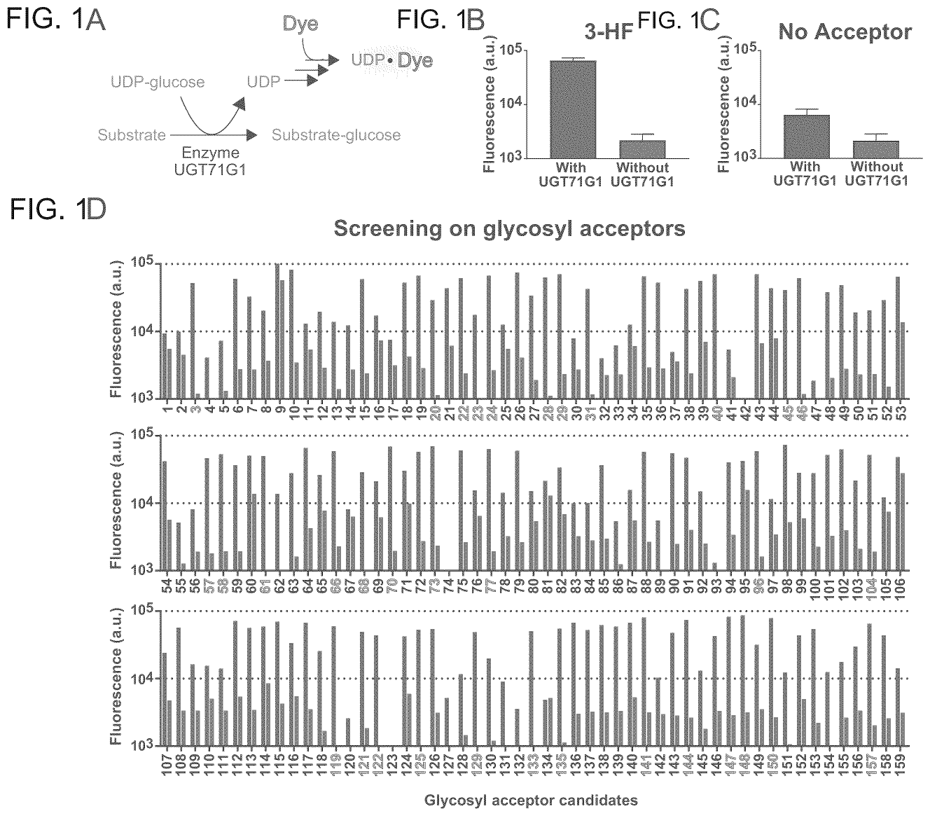

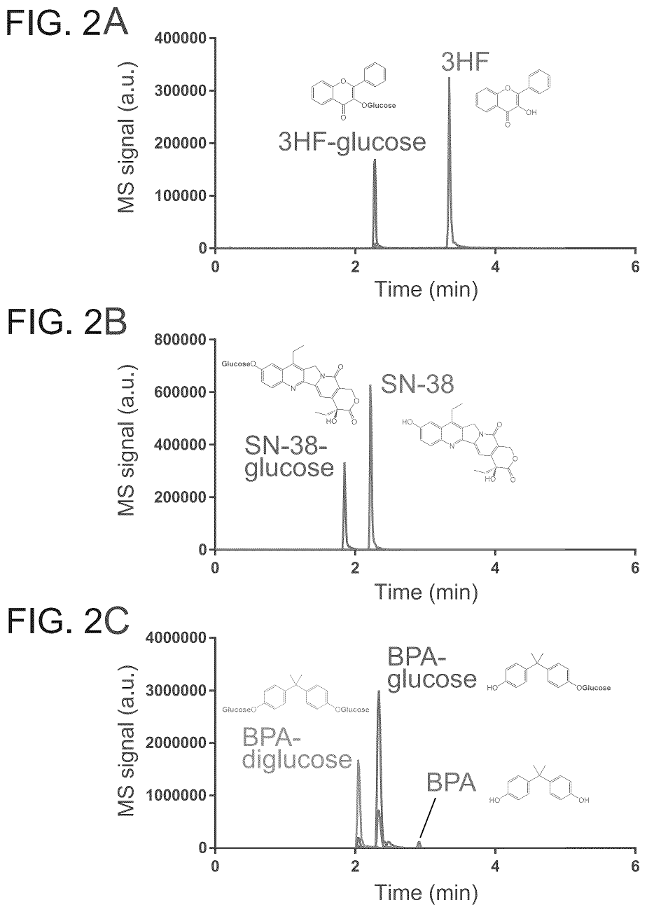

FIG. 1C depicts the results of the assay performed in the absence of a glycosyl acceptor, aiming to determine whether the florescence signal increase is due to the UGT activities or not.

FIG. 1D provides the results of assays used to test 159 compounds using the methods of the present disclosure.

FIG. 2A are chromatograms characterizing the substrates and products of UGT71G1 reacting with 3-hydroxyflavone (3HF).

FIG. 2B are chromatograms characterizing the substrates and products of UGT71G1 reacting with 7-ethyl-10-hydroxy-camptothecin (SN-38)

FIG. 2C are chromatograms characterizing the substrates and products of UGT71 G1 reacting with bisphenol A (BPA).

### DETAILED DESCRIPTION

The following discussion is directed to various exemplary aspects. However, one skilled in the art will understand that the examples disclosed herein have broad application, and that the discussion of any aspect is meant only to be exemplary of that aspect, and not intended to suggest that the scope of the disclosure, including the claims, is limited to that aspect.

Disclosed herein are one or more methods for the high-throughput detection of enzymatic activity. In an aspect, the present disclosure comprises a method of detecting UGT-transferase activity by carrying out an assay to detect free UDP comprising contacting (i) a UGT-transferase; (ii) a UDP-sugar conjugate; and (iii) a probe to form a mixture and subjecting the mixture to conditions suitable for the release of UDP, also termed the formation of “free UDP”. A method of the present disclosure further comprises contacting the reacted mixture with one or more UDP-reactive dyes and quantifying the level of free UDP present in the mixture. The assay is depicted schematically in FIG. 1A where a UDP-sugar (UDP-glucose) is reacted with a substrate in the presence of a UGT-transferase to form free UDP. The free UDP is then detected by reaction with a UDP-reactive dye.

In one or more aspects, an assay mixture of the present disclosure comprises a UDP-glucuronosyltransferase (UGT) (EC:2.4.1.17). UGT-transferases are utilized during Phase II metabolism to increase the hydrophilicity of a xenobiotic (e.g., drug) which facilitates transport and elimination of the xenobiotic. The UGT-transferase family of enzymes catalyzes a glucuronidation reaction which consists of the transfer of the sugar group from a uridine 5′-diphospho-sugar to substrate molecules that contain oxygen, nitrogen, sulfur or carboxyl functional groups. The reaction also produces free UDP. UGT-transferases are enzymes that represent a highly responsive defense system against the mutagenicity of carcinogens and the toxicity of both xenobiotics and endogenous metabolic intermediates. In one or more aspects, any UGT-transferase capable of catalyzing the transfer of a sugar from a UDP-sugar conjugate to a substrate may be utilized in the present disclosure. A UGT-transferase for use in the present disclosure being “capable of catalyzing” a certain reaction indicates that a UGT-transferase is capable of catalyzing transfer of a sugar group from a sugar donor to a substrate (also termed an acceptor). It is to be understood that under appropriate reaction conditions the rate at which the reaction product (in the example the adduct of the sugar group and the acceptor) is formed is at least 10-fold higher in the presence of said UGT-transferase than the rate at which the reaction product is formed in a control experiment in the absence of said UGT-transferase.

In one or more aspects, an assay mixture of the present disclosure comprises a UDP-sugar conjugate. Nonlimiting examples of UDP-sugar conjugates suitable for use in the present disclosure include UDP-glucose, UDP-glucuronic acid and UDP-rhamnose UDP-xylose, UDP-glucuronic acid, UDP-arabinose, UDP-rhanmose, UDP-galactose, GDP-fructose and GDP-mannose. In an aspect, the UDP-sugar conjugate is UDP-glucose.

In one or more aspects, an assay mixture of the present disclosure comprises a probe. Herein a probe refers to a molecule that may be capable of being conjugated with the sugar molecule of the UDP-sugar conjugate. In the event that the probe is found to conjugate with the sugar molecule, the probe is now considered a substrate. Nonlimiting examples of probes suitable for use in the present disclosure include bile acids, bilirubin, steroid hormones, drugs such as acetaminophen, morphine, and tamoxifen, environmental toxins, dietary components like flavonoids, and endogenous compounds such as thyroid hormones. In one or more aspects, the probe is a xenobiotic or an endogenous molecule with suitable acceptor functional groups for glucuronidation. In other aspects, the probes utilized in the assay mixture may not be a suitable UGT-transferase substrate.

In one or more aspects, the assay mixture comprises a UGT-transferase present in an amount of from about μg to about 10 μg; a UDP-sugar conjugate in an amount of from about 1 nmol to about 10 nmol; and a probe present in an amount of from about 1 nmol to about 10 nmol. The assay mixture may further include a buffered media suitable to provide a pH of from about 6.5 to about 7.5. The reaction mixture may be subject to reaction conditions that include one or more of the following: a reaction temperature of from about 30° C. to about 40° C.; and a reaction time period of from about 30 minutes to about 60 minutes. The reacted mixture may then be contacted with a UDP-reactive dye.

Herein a UDP-reactive dye is a molecule that can bind to free UDP and provide a spectroscopic indication that is proportional to the amount of associated free UDP. In one or more aspects, the UDP-reactive dye is fluorescent and the fluorescence is altered when associated with the free UDP. In one or more aspects, the UDP-reactive dye does not bind UDP unless it is a “free” molecule and not associated with a sugar. Nonlimiting examples of UDP-reactive dyes suitable for use in the present disclosure include 1, 8-naphthylimide-based; 2-naphthyl (2-naphthylaminophenyl) imidazole; 2-(6-(2-(N,N-dimethylaminoethylamino)-1,3-dioxo-1H-benzo[de]isoquinolin-2(3H)-yl)ethyl methacrylate (NI1); 2-(6-(2-(N,N-dimethylaminoethoxy)-1,3-dioxo-1H-benzo[de]isoquinolin-2(3H)-yl)ethyl methacrylate; perylene-based probe; perylene; perylene diimides (PDIs); benzo[ghi]perylene; 3,4,9,10-perylenetetracarboxylic dianhydride (PTCDA), and various substituted perylene derivatives with different functional groups attached to the perylene core.

In alternative aspects, the UDP-reactive dye exhibits a spectroscopic response when associated with a UDP-sugar conjugate that differs from the spectroscopic response observed the UDR-reactive dye is associated with a free UDP. For example, a UDP-reactive dye may have an emission wavelength x when unbound and an emission wavelength y when associated with a free UDP. In other aspects, the UDP-reactive dye has an emission wavelength z when associated with a UDP-sugar conjugate. In one or more aspects, x≠y≠z.

In one or more aspects, the UDP-reactive dye is introduced to the reacted mixture in an amount of from about 1 nmol to about 10 nmol, and a temperature of from about 30° C. to about 40° C. The result is a final mixture that may be analyzed spectroscopically.

The exact concentrations of free UDP in the reacted mixture may be determined by generating a calibration curve using the presently disclosed assay and known concentrations of UDP. In one or more aspects, the amount of free UDP generated by the UGT-transferase is used to designate whether the probe is a suitable UGT-transferase substrate.

In one or more aspects, the methods disclosed herein may be used to develop a high throughput screen (HTS) assay. For example, the HTS assay may be run in a microplate format. Assays that can be run in a microplate format generally benefit from the ability to use relatively low volumes of reagents in a single assay, the ability to scale assay volume as desired, and the ability to increase throughput (the number of assays that can be run at one time).

Disclosed herein is an HTS that was developed to screen for substrates of uridine-glycosyltransferases that uses, in one aspect, UDP-glucose as a co-substrate. As this method is based on detecting the release of UDP from glucose, it is independent from the glycosyl acceptor, which allows its utilization for screening a wide range of substrate candidates. Comparing to traditional method for characterizing UGT activities that requires LC-MS/MS techniques, the presently disclosed fluorescent assay requires less time, labor, and instrumentation costs, which facilitates the screening of an extensive set of compounds for studying substrate specificity.

The presently disclosed screening method can be applied to a broad range of UGTs such as UDP-glycosyltransferases to characterize their substrate profile. Based on the UniProt database, over 40,000 UDP-glycosyltransferases have been documented and about 400 UGTs have been verified on protein level. There is a great potential to harness these enzymes as tools to metabolize toxic species. The presently disclosed high-throughput assay opens these opportunities.

### EXAMPLES

The presently disclosed subject matter having been generally described, the following examples are given as particular aspects of the subject matter and to demonstrate the practice and advantages thereof. It is understood that the examples are given by way of illustration and are not intended to limit the specification or the claims in any manner.

In this study, a high-throughput fluorescent assay was developed for detecting enzymatic activities of UDP-glycosyltransferases (UGTs), which transfer the glucosyl moiety from UDP-glucose to an acceptor molecule. To test the validity of the method design, substrates for a plant UGT homology, UGT71 G1, from Medicago truncatula was screened. This homolog was selected because it has a broad substrate recognition; previous studies showed that UGT71G1 glycosylates multiple hydroxyl groups at various natural products, such as genistein and biochanin A9. Additionally, UGT71G1 is a cytosolic enzyme that remains active when expressed in bacterial cells. These properties render this UGT homology to have great potential in metabolic applications.

With the high-throughput assay developed 159 samples of known substrates/non-substrates of human UGTs (including endogenous substrates, drugs and metabolites, natural products, and environmental/dietary toxins) for UGT71G1 activities were screened. Among positive candidates in the screening results, a LC-MS/MS method was used to confirm glycosylated products from three xenobiotic substrates with potential medical and environmental applications, including an environmental pollutant from plastic, bisphenol A (BPA) and an active and toxic metabolite of anticancer drug irinotecan, 7-Ethyl-10-hydroxycamptothecin (SN-38). Together, the present disclosure supports the use of the disclosed high-throughput method for discovering new substrates of UGT homologs and it highlights the potential of UGT71G1 for a wide range of applications.

### Materials and Methods

Materials. E. coli BL21(DE3) cells containing pET28a-UGT71 G1 were prepared in previous studies. LB medium broth, inducers, and antibiotics were obtained from VWR (Radnor, PA, USA).

All drugs and chemicals were sourced from either Sigma-Aldrich (Saint Loise, MO), INDOFINE Chemical Company (Hillsborough Township, NJ), or Toronto research chemicals (Toronto, Canada). The MS grade water, methanol, and acetonitrile were obtained from EMD (Gibbstown, NJ, USA).

### Protein Expression and Purification of UGT71G1.

The enzyme UGT71 G1 was expressed in Escherichia coli strain BL21 (DE3) and purified as described previously with some modification. Briefly, E. coli BL21(DE3) cells transformed with the plasmid pET28a-UGT71G1 were grown at 37° C. in Luria-Bertani medium containing 50 mg/mL kanamycin until OD600 nm reached 0.6 to 0.8. The UGT71G1 protein expression was induced with 0.5 mM isopropyl 1-thio-β-galactopyranoside for overnight at 16° C. Cells were pelleted and resuspended in lysis buffer (50 mM Tris-HCl, pH 8.0, 500 mM NaCl, 10 mM imidazole, and 10 mM β-mercaptoethanol), and lysed by sonication on ice. After centrifugation at 12,000 rpm at 4° C. for 20 min, Ni²⁺-NTA agarose was added to the supernatant containing the target proteins. After incubation for 40 to 60 min, the mixture was transferred into a disposable column and washed extensively with lysis buffer (˜100 column volumes). The His-tagged proteins were eluted with elution buffer (50 mM Tris-HCl, pH 8.0, 500 mM NaCl, 250 mM imidazole, and 10 mM β-mercaptoethanol). The protein was further purified on a SUPERDEX-200 gel filtration column (GE Healthcare) and concentrated to ˜6 mg/mL in 10 mM NaCl, 10 mM Tris-HCl, pH 7.5, and 5 mM β-mercaptoethanol.

High-throughput fluorescent assay for screening candidates of UGT glycosylation. Characterization of UDP formation involved the MicroMolar UDP assay kit (ProFoldin). For each reaction, reagents used from this kit include 3 μL of 10× buffer, 0.3 μL of 100×MUD reagent 1, and 0.3 μL of 100×MUD reagent 2. These reagents were mixed with 6 μg of purified enzyme UGT71G1 (this enzyme was not added for each negative control), the substrate candidate (final concentration ranging from 0.8 to 160 μM based on substrate solubility), UDP-glucose (50 μM), and ultrapure water to reach a final volume of 30 μL. The reaction mixture was incubated at 37° C. for 45 minutes on a 384-well microtiter plate. Each sample was then mixed with 30 μL of 1× fluorescence dye from the MICROMOLAR UDP assay kit and fluorescence was measured immediately with a BIOTEK SYNERGY H1 M florescence microplate reader at excitation 485 nm and emission 535 nm.

Liquid chromatography-coupled mass spectrometric analysis of substrates and products. The glycosylation reaction for plant UGT71G1 enzyme extracted from E. coli BL21(DE3) cells containing UGT71G1-pET28a was performed similar to the glucuronidation reaction as those published previously 17 with minor modification. Briefly, the enzyme UGT71G1 (final concentration 0.05-0.5 μg/ml) was mixed with UDP-glucose (50 μM) and substrate (final concentration 10 μM) in 50 mM potassium phosphate buffer (pH 7.4) to reach a final volume of 200 μL. This reaction mixture was incubated at 37° C. and at various time points for each substrate to obtain the highest product-to-substrate ratio: 2 hours for 3HF, 4 hours for SN-38, and 24 hours for BPA; reactions were ended with the addition of 200 μL methanol. The samples were centrifuged at 14000 rpm for 15 mins and supernatant was subjected to UPLC-MS/MS system for analysis after appropriate dilution with 50% MeOH solution.

LC-MS/MS analysis was performed with an EXIONLC™ UHPLC system coupled with API 5500 Q-Trap triple quadrupole mass spectrometer. A BEH C18 column (50×2.1 mm I.D., 1.7 μm, Waters, Milford, MA, USA) was used for liquid chromatography. For mobile phase, mobile phase A (MPA) was 0.1% formic acid in water and mobile phase B (MPB) was 100% acetonitrile; flow rate was 0.45 mL/min with a column temperature of 45° C. An amount of 10 μL sample was injected for each analysis and it was eluted with the following mobile phase profile: 0-0.5 min, 5-10% MPB, 0.5-1.5 min, 10-40% MPB, 1.5-4.0 min, 40-90% MPB, 4.0-4.4 min, 90% MPB, 4.4-4.6 min, 90-5% MPB, 4.6-5.0 min, 5% MPB. Gradient mobile phase for mycophenolic acid and metabolites: 0-1.0 min, 5-20% MPB, 1.0-1.5 min, 20% MPB, 1.5-4.0 min, 20-40% MPB, 4.0-5.0 min, 40-90% MPB, 5.0-5.5 min, 90-5% MPB, 5.5-6.0 min, 5% MPB.

For a high-through method to robustly detect UGT activities with different glycosyl acceptor, it is necessary to target a change that is independent from the acceptor molecule. UDP-glycosyltransferases take UDP-glucose as a co-substrate; while the glucosyl moiety is transferred to the acceptor molecule, UDP is released as a by-product (FIG. 1A). Thus, UGT activities can be detected by the rise in UDP level in the reaction mixture. In our method to monitor an increase in UDP concentration, the UGT71G1 enzymatic reaction was coupled to the UDP assay kit, in which the release of UDP leads to an increase in fluorescence intensity.

With our high-throughput method, 159 samples were investigated as glycosyl acceptors, which include 143 compounds and some replicates of these candidates from different sources. The xenobiotic, 3-hydroxyflavone (3HF), is one of the candidates that generated a significant increase in fluorescence intensity when tested with the abovementioned assay. To determine whether the rise in fluorescence intensity is due to UGT activities, we also performed the assay in the absence of enzyme UGT71 G1, evaluating the background fluorescent signals from the compound sample. As shown in FIG. 1B, the presence of both 3HF and UGT71G1 led to a 30-fold increase in fluorescent signal, comparing to the condition without UGT71G1. Similarly, the detection method was evaluated in the absence of glycosyl acceptor (FIG. 1C). The presence of enzyme UGT71 G1 alone caused a 3-fold increase in the signal, which suggested that the isolated UGT71G1 contained contaminating hydrolases that non-specifically hydrolyze UDP-glucose, generating a basal level of UDP.

Among the 143 compounds that were screened with the high-throughput assay, they were selected based on their structural similarity to other known substrates of human UGT enzymes. This compound library is shown in Supplementary Table 1. Each compound was assayed in both the presence and absence of enzyme UGT71 G1 (FIG. 1D). To test the robustness of this assay, 13 compounds were run in duplicate and 1 compound (3-hydroxy-6-methoxyflavone; compound 24) was run 4 times; replicates of the same compound were from different stocks.

Among those 144 compounds, 31 of them were designated strong candidates as glycosyl acceptor, in which they generated an over 25-fold increase in florescence intensity with UGT71G1 (FIG. 1D and Supplementary Table 2). Most of these candidates are natural plant flavonoids (including 7 flavones, 8 flavonols, 2 flavonones, 3 isoflavones, 2 chalcones, and 1 anthocyanin) and they are potentially among those natural substrates of UGT71G1. A few other strong candidates are xenobiotics, including drug metabolite, SN-38 (compound 58) and environmental toxin, bisphenol A (BPA; compound 135). Both of these two candidates are cytotoxic and thus, they can be targets for health-related applications. SN-38 is the active metabolite of a chemotherapeutic agent, irinotecan; among patients that administer irinotecan, SN-38 is excreted into the intestines via bile and leads to 25% of these patients to experience diarrhea. For BPA, it is broadly used in the manufacturing of plastics, which leads to its spread in the environment. Many studies have shown that BPA affects the development of brain and prostate glands. Therefore, using UGT71G1 to derivatize these compounds may provide an efficient means to reduce human exposures to them. Other strong candidates include coumarin (1), smaller phenols (3), polyphenol (1), anthraquinone (1), and microbial metabolite of proanthocyanidin (1). Interestingly, substrates of UGT71 G1 ranged from compounds with single hydroxy group in structure to multiple hydroxy groups (e.g., cyanidin, Isorhamnetin), and could be glycosylated at multiple hydroxyl positions (only confirmed with one compound, diglucose of bisphenol A (FIG. 1C), however, there was no obvious structure activity relationship that can be drawn from the results.

Among the rest of the glycosyl acceptor candidates, most of them produced low fluorescence signals in both the presence and absence of UGT71G1, implying that they do not serve as effective substrates for this enzyme in this assay condition. However, several compounds led to significant levels of fluorescence even in the absence of UGT71G1, suggesting that they either directly cause the release of UDP or interact with the fluorescent dye. The most representative compounds in this category includes sorafenib (compound 9), 7,2′-dihydroxy-flavone (compound 53), 5,4′-dihydroxyflavone (compound 60), and 7-hydroxy-4′-methoxy-flavone (compound 106). As a result, our method cannot accurately assess the efficiency of UGT activities on these candidates.

Confirmation of UDP-Glycosyltransferase Activities with LC-MS/MS

To validate our results from the high-throughput fluorescent assay, a liquid chromatography-coupled tandem mass spectrometric (LC-MS/MS) method was used to monitor the reaction of UGT71G1 with 3-hydroxyflavone, bisphenol A, and SN-38. Table 1 shows transitions that are used to monitor these substrates and their products in our LC-MS/MS method.

TABLE 1
Transitions in tandem mass spectrometric
method for monitoring UGT71G1 activities.

| | Parental | Fragmented | | | |
|---|---|---|---|---|---|
| | ion Q1 | ion Q3 | DP^{a} | CE^{b} | CXP^{c} |
| Compound | (m/z) | (m/z) | (V) | (V) | (V) |
| 3HF | 239 | 165 | 59 | 42 | 21 |
| 3HF-Glucose | 401 | 239 | 59 | 42 | 21 |
| SN-38 | 393 | 349 | 5 | 33 | 25 |
| SN-38-Glucose | 555 | 393 | 5 | 33 | 25 |
| BPA | 227 | 133 | −157 | −38 | −11 |
| BPA-Glucose | 389 | 227 | −157 | −38 | −11 |
| BPA-Diglucose | 551 | 227 | −157 | −38 | −11 |

Note
^{a}DP: declustering potential
^{b}CE: collision energy
^{c}CXP: collision cell exit potential
These machine settings affect fragment compositions of MS/MS analyses

Analytical standards were used to confirm the elution of three xenobiotic substrates, which formed those peaks labeled in FIGS. 2A-2C. After reaction with the enzyme UGT71G1 in the presence of co-substrate UDP-glucose, at least one new peak was formed in the LC-MS/MS analysis of the three substrate samples and the m/z values match the assigned structure of 0-glucose conjugate(s) as shown.

For 3-hydroxyflavone (FIG. 2A), it eluted at 3.34 min and the metabolite peak eluted at 2.28 min with a parental ion m/z value of 401, which is the expected m/z value of a 3-hydroxyflavone with a glucose on its original hydroxyl group. After collision-induced dissociation (MS/MS), the major fragment has an m/z value the same as that of 3-hydroxyflavone (Q1/Q3 transition masses 401/239). Similarly, SN-38 (FIG. 2B) eluted at 2.22 min and its glucose derivative eluted at 1.85 min with expected m/z for the parental ion and the fragmented ion (the fragmented ion's m/z was the same as that of SN-38; Q1/Q3 transition masses 555/393). For BPA (FIG. 2C), reaction catalyzed by UGT71G1 led to two products. The major product had a parental ion with m/z value as a BPA with one hydroxyl group glycosylated (elution time was 2.32 min) (Q1/Q3 transition masses 389/227). The minor product was expected to be a BPA with both of its hydroxyl group glycosylated (Q1/Q3 transition masses 551/227) and its eluted earlier than the monoglycosylated product (2.04 min). These results strongly support that the three analyzed xenobiotics are substrates of UGT71G1 for glycosylation.

With the disclosed method, a set of potential substrates for a plant UGT homolog, UGT71 G1, was discovered and three of them were validated, including 3HF, BPA, and SN-38. 3HF is a synthetic flavonoid that is not found naturally and thus, glycosylation of this compound may not provide any substantial benefits. In contrast, a robust method for derivatizing BPA and SN-38 can lead to significant biomedical applications as these two compounds are related to toxicity-induced health issues. BPA can be released from polycarbonate plastics and its presence is now ubiquitous in the environment. Recent studies support that BPA may induce carcinogenesis and mutagenesis. This xenobiotic may also generate adverse health effects on brain development, potentially affecting children's behavior. SN-38 is a cytotoxic metabolite of the chemotherapeutic agent, irinotecan; when it is excreted to the gut via bile, it can lead to a range of gastrointestinal side effects, such as severe delayed onset diarrhea. If glycosylation of these xenobiotics reduces their toxicity, it can provide a new means to improve human health.

The plant UGT homology, UGT71G1, has the potential for biomedical and environmental applications. For many well-studied UGT homologs, such as the human UGT1A1, are membrane proteins, which are difficult to express and purify. For the plant UGT71G1, it is a highly soluble cytosolic protein, and it is active when expressed in bacteria. These properties facilitate its use in many settings for derivatizing target species, such as in the digestive system or natural environments.

A list of 159 samples as candidates of UGT71 G1 substrate. Table S2: Potential UGT71 G1 substrates based on fluorescent assay screening.

TABLE S1
A list of 159 samples as candidates of UGT71G1 substrate

| Sample No. | Cas No. | Name | Chemical Class |
|---|---|---|---|
| 1 | 183321-74-6 | Erlotinib | Anticancer drug |
| 2 | 319460-85-0 | Axitinib | Anticancer drug |
| 3 | 6068-76-4 | 3,2′-Dihydroxy-flavone | Flavonol |
| 4 | 529-44-2 | Myricetin | Flavone |
| 5 | 263407-43-8 | 6,3′,4′-Trihydroxy flavone | Flavone |
| 6 | 93-35-6 | Umbelliferone | Coumarin |
| 7 | 15236-07-4 | 5,7-Dimethoxy-3- | Flavonol |
| | | hydroxyflavone | |
| 8 | 140439-35-6 | 6-Hydroxy-3′- | Flavone |
| | | methoxyflavone | |
| 9 | 284461-73-0 | Sorafenib | Anticancer drug |
| 10 | 35244-11-2 | 2′-Hydroxyflavone | Flavone |
| 11 | 491-78-1 | 5-hydroxyflavone | Flavone |
| 12 | 63046-09-3 | 6,4′-Dihydroxyflavone | Flavone |
| 13 | 2150-11-0 | 7,3′,4′-Trihydroxyflavone | Flavone |
| 14 | 520-28-5 | Techtochrysin | Flavone |
| 15 | 632-85-9 | Wogonin | Flavone |
| 16 | 35794-88-8 | 6-Hydroxy-4′- | Flavone |
| | | methoxyflavone | |
| 17 | 51-43-4 | Epinephrine | Hormone/Neurotransmitter |
| 18 | 6665-86-7 | 7-Hydroxyflavone | Flavone |
| 19 | 492-00-2 | 3,7-Dihydroxyflavone | Flavonol |
| 20 | 4143-64-0 | 3′,4′-Dihydroxyflavone | Flavone |
| 21 | 73046-40-9 | 5,7,2′-Trihydroxyflavone | Flavone |
| 22 | 480-41-1 | Naringenin | Flavonone |
| 23 | 518-82-1 | Emodin | Anthraquinone |
| 24 | 93176-00-2 | 3-Hydroxy-6- | Flavonol |
| | | methoxyflavone | |
| 25 | 50-28-2 | Estradiol | Steroid Hormone |
| 26 | 577-85-5 | 3-Hydroxyflavone | Flavonol |
| 27 | 14919-49-4 | 3,4′-Dihydroxyflavone | Flavonol |
| 28 | 480-19-3 | Isorhamnetin | Flavonol |
| 29 | 93176-00-2 | 3-Hydroxy-6- | Flavonol |
| | | methoxyflavone | |
| 30 | 103-90-2 | Acetaminophen | Drug |
| 31 | 548-83-4 | Galangin | Flavonol |
| 32 | 120889-04-5 | 2-Hydroxy-1-methyl-6- | Heterocyclic amine |
| | | phenylimidazo[4,5- | |
| | | b]pyridine | |
| 33 | 62-31-7 | Dopamine HCl | Hormone/Neurotransmitter |
| 34 | 53-16-7 | Estrone | Steroid Hormone |
| 35 | 108238-41-1 | 3,6-Dihydroxyflavone | Flavonol |
| 36 | 55977-09-8 | 3,3′-Dihydroxyflavone | Flavonol |
| 37 | 108238-40-0 | 7,3′-Dihydroxyflavone | Flavone |
| 38 | 58-22-0 | Testasterone | Steroid Hormone |
| 39 | 490-46-0 | Epicatechin | Catechin |
| 40 | 528-58-5 | Cyanidin | Anthocyanin |
| 41 | 105650-23-5 | 2-Amino-1-methyl-6- | Dietary Carcinogen |
| | | phenylimidazo[4,5- | |
| | | b]pyridine (PhIP) | |
| | | Hydrochloride | |
| 42 | 557795-19-4 | Sunitinib | Anticancer drug |
| 43 | 480-40-0 | Chrysin | Flavone |
| 44 | 2196-14-7 | 4′,7-Dihydroxyflavone | Flavone |
| 45 | 38183-03-8 | 7,8-Dihydroxyflavone | Flavone |
| 46 | 151698-64-5 | 3,7,3′-Trihydroxyflavone | Flavonol |
| 47 | 60-82-2 | Phloretin | Chalcone |
| 48 | 2107-78-0 | 3,4-Dimethyl umbelliferone | Coumarin |
| 49 | 621-54-5 | 3-(3- | Microbial Metabolite of |
| | | Hydroxyphenyl)propionic | proanthocyanidins |
| | | acid | |
| 50 | 70460-18-3 | 3′-Hydroxyflavone | Flavone |
| 51 | 6665-83-4 | 6-Hydroxyflavone | Flavone |
| 52 | 2034-65-3 | Resokaempferol | Flavonol |
| 53 | 77298-66-9 | 7,2′-Dihydroxyflavone | Flavone |
| 54 | 61546-59-6 | 6-Hydroxy-2′- | Flavone |
| | | methoxyflavone | |
| 55 | 989-51-5 | Epigallocatechin Gallate | Catechin |
| 56 | 2086-83-1 | Berberine | Alkaloid |
| 57 | 501-97-3 | 3-(4- | Microbial Metabolite of |
| | | Hydroxyphenyl)propionic | proanthocyanidins |
| | | acid | |
| 58 | 86639-52-3 | SN-38 | Anticance drug metabolite |
| 59 | 4143-63-9 | 4′-hydroxyflavone | Flavone |
| 60 | 6665-67-4 | 5,4′-Dihydroxyflavone | Flavone |
| 61 | 38183-04-9 | 6,7-Dihydroxyflavone | Flavone |
| 62 | Not known | 6,7,3′-Trihydroxyflavone | Flavone |
| 63 | 520-18-3 | Kaempferol | Flavonol |
| 64 | 32272-23-4 | 4′-Hydroxy-7- | Flavone |
| | | methoxyflavone | |
| 65 | 520-28-5 | Techtochrysin | Flavone |
| 66 | 520-33-2 | Hesperetin | Flavonone |
| 67 | 3952-18-9 | Cupressuflavone | Biflavonoid |
| 68 | 6068-78-6 | 3,3′,4′-Trihydroxyflavone | Flavonol |
| 69 | 106848-87-7 | 4′-Hydroxy-5- | Flavone |
| | | methoxyflavone | |
| 70 | 1214-47-7 | 2′-hydroxychalcone | Chalcone |
| 71 | 62507-01-1 | 3′-benzyloxy-5,7-dihydroxy- | Flavone |
| | | 3,4′-dimethyoxy flavone | |
| 72 | 6665-86-7 | 7-Hydroxyflavone | Flavone |
| 73 | 40316-76-5 | 8-Hydroxy-7- | Flavone |
| | | methoxyflavone | |
| 74 | 149732-51-4 | Demethyl curcumin | Polyphenol |
| 75 | 20426-12-4 | 4-hydroxychalcone | Chalcone |
| 76 | 253195-19-6 | 3,6,4′-Trihydroxyflavone | Flavonol |
| 77 | Not known | 4′-Hydroxy-3′- | Flavone |
| | | methoxyflavone | |
| 78 | 656229-81-1 | 4′,5-Di-O-acetyl Genistein | Isoflavone |
| 79 | 40957-83-3 | Glycitein | Isoflavone |
| 80 | 520-36-5 | Apigenin | Flavone |
| 81 | 3450-72-4 | 3,4-Diphenyl-7- | Coumarin |
| | | hydroxycoumarin | |
| 82 | 6068-78-6 | 2′,3′-Dimethoxy-3- | Flavonol |
| | | hydroxyflavone | |
| 83 | 2196-14-7 | 7,4′-Dihydroxyflavone | Flavone |
| 84 | 13252-83-0 | 4-Hydroxy-6- | Coumarin |
| | | methylcoumarin | |
| 85 | Not known | 6-hydroxy-7- | Flavone |
| | | methoxyflavone | |
| 86 | 93322-61-3 | 3,7-Dihydroxy-3′,4′- | Flavonol |
| | | dimethoxyflavone | |
| 87 | 18651-15-5 | 7-hydroxy-3-methylflavone | Flavone |
| 88 | 446-72-0 | Genistein | Isoflavone |
| 89 | 528-48-3 | Fisetin | Flavonol |
| 90 | 644-78-0 | 2-Hydroxychalcone | Chalcone |
| 91 | 6665-81-2 | 3-hydroxy-5- | Flavone |
| | | methoxyflavone | |
| 92 | 531-95-3 | (+/−)-Equol | Isoflavone |
| 93 | 6151-25-3 | Quercetin dihydrate | Flavonol |
| 94 | 4002-52-2 | 4′-hydroxy-6- | Flavone |
| | | methoxyflavone | |
| 95 | 15235-99-1 | 7-hydroxy-5-methylflavone | Flavone |
| 96 | 501-36-0 | Resveratrol | Polyphenol |
| 97 | 961-29-5 | Isoliquiritigenin | Chalcone |
| 98 | 76666-32-5 | 3-Hydroxy-3′- | Flavonol |
| | | methoxyflavone | |
| 99 | 263365-35-1 | 3(2′-Chlorophenyl)-7- | Coumarin |
| | | hydroxy-4-phenylcoumarin | |
| 100 | 5631-67-4 | 7-hydroxychromanone | Phenol |
| 101 | 2657-25-2 | 4′-hydroxychalcone | Chalcone |
| 102 | 486-66-8 | Daidzein | Isoflavone |
| 103 | 6889-78-7 | 3-Hydroxy-4′- | Flavonol |
| | | methoxyflavone | |
| 104 | Not known | 7-Hydroxy-6- | Isoflavone |
| | | methoxyisoflavone | |
| 105 | 19202-36-9 | Hinokiflavone | Biflavonoid |
| 106 | 487-24-1 | Pratol | Flavone |
| 107 | 552-59-0 | Prunetin | Isoflavone |
| 108 | 491-54-3 | Kaempferide | Flavonol |
| 109 | 93097-20-2 | 3-Hydroxy-4′-methoxy-6- | Flavone |
| | | methylflavone | |
| 110 | Not known | 3-Hydroxy-6-methyl-3′,4′- | Flavone |
| | | methylenedioxyflavone | |
| 111 | 489-32-7 | Icariin | Polyphenol |
| 112 | 6971-18-2 | 3-Hydroxy-6-methylflavone | Flavonol |
| 113 | 7478-60-6 | 3-Hydroxy-7- | Flavonol |
| | | methoxyflavone | |
| 114 | Not known | 6,3′-Dimethoxy-3- | Flavonol |
| | | hydroxyflavone | |
| 115 | 93176-00-2 | 3-Hydroxy-6- | Flavonol |
| | | methoxyflavone | |
| 116 | 93097-20-2 | 3-Hydroxy-3′4′-dimethoxy- | Flavonol |
| | | 6-methylflavone | |
| 117 | 13198-99-7 | 7,4′-dimethoxy-3- | Flavonol |
| | | hydroxyflavone | |
| 118 | 520-34-3 | Diosmetin | Flavone |
| 119 | 29219-03-2 | 3-Hydroxy-2′- | Flavonol |
| | | methoxyflavone | |
| 120 | 60-82-2 | Phloretin | Chalcone |
| 121 | 632-85-9 | Wogonin | Flavone |
| 122 | 548-83-4 | Galangin | Flavonol |
| 123 | 529-44-2 | Myricetin | Flavonol |
| 124 | 490-46-0 | (−)-Epicatechin | Catechin |
| 125 | 93-35-6 | 7-hydroxycoumarin | Coumarin |
| 126 | 480-40-0 | Chrysin | Flavone |
| 127 | 105650-23-5 | 2-Amino-1-methyl-6- | Dietary Carcinogen |
| | | phenylimidazo[4,5- | |
| | | b]pyridine (PhIP) | |
| | | Hydrochloride | |
| 128 | 491-78-1 | 5-hydroxyflavone | Flavone |
| 129 | 93176-00-2 | 3-hydroxy-6- | Flavonol |
| | | methoxyflavone | |
| 130 | 491-80-5 | Biochanin A | Isoflavone |
| 131 | 2078-54-8 | Propofol | Drug |
| 132 | 802918-57-6 | Silybin | Polyphenol |
| 133 | 480-19-3 | Isorhamnetin | Flavonol |
| 134 | 485-72-3 | Formononetin | Isoflavone |
| 135 | 80-05-7 | Bisphenol A | Environmental Toxin |
| 136 | 17834-02-5 | 6-Hydroxywarfarin | Drug metabolite |
| 137 | 2035-15-6 | Maackiain | lisoflavone |
| 138 | 17834-03-6 | 7-Hydroxywarfarin | Drug metabolite |
| 139 | 18651-15-5 | 7-hydroxy-3-methylflavone | Flavone |
| 140 | Not known | 7-hydroxy-2′- | Flavone |
| | | methoxyflavone | |
| 141 | 106-41-2 | 4-bromophenol | Phenol |
| 142 | 83219-99-2 | 10-Hydroxywarfarin | Drug metabolite |
| 143 | 6068-78-6 | 2′,3′-Dimethoxy-3- | Flavone |
| | | hydroxyflavone | |
| 144 | 17238-05-0 | Dihydrodaidzein | Isoflavone |
| 145 | 51059-44-0 | Wogonoside | Flavone Glycoside |
| 146 | 15236-07-4 | 5,7-Dimethoxy-3- | Flavone |
| | | hydroxyflavone | |
| 147 | 552-59-0 | Prunetin | Isoflavone |
| 148 | 100-02-7 | Nitrophenol | Phenol |
| 149 | 153-18-4 | Rutin | Flavonol Glycoside |
| 150 | 480-11-5 | Oroxylin A | Flavone |
| 151 | 32520-55-1 | 6-Methoxy-5,7,3′,4′- | Flavone |
| | | tetrahydroxyflavone | |
| 152 | 53152-21-9 | Buprenorphine | Drug |
| 153 | 18103-42-9 | 5,7-Dihydroxy-3′,4′,5′- | Flavone |
| | | trimethoxyflavone | |
| 154 | 82640-04-8 | Raloxifene | Drug |
| 155 | 3681-93-4 | Vitexin | Flavone glycoside |
| 156 | 33429-83-3 | 3,4′-Dimethoxy-5,7,3′- | Flavone |
| | | trihydroxyflavone | |
| 157 | 622-62-8 | 4-Ethoxyphenol | Phenol |
| 158 | 24280-93-1 | Mycophenolic acid | Immunosuppressant drug |
| 159 | 153-98-0 | Serotonin | Neurotransmitter |

TABLE S2
Potential UGT71G1 substrates based on fluorescent assay screening

| Sample No. | Cas No. | Name | Chemical Class |
|---|---|---|---|
| 3 | 6068-76-4 | 3,2′-Dihydroxy flavone | Flavonol |
| 20 | 4143-64-0 | 3′,4′-Dihydroxyflavone | Flavone |
| 22 | 480-41-1 | Naringenin | Flavonone |
| 23 | 518-82-1 | Emodin | Anthraquinone |
| 24 | 93176-00-2 | 3-Hydroxy-6- | Flavonol |
| | | methoxyflavone | |
| 28 | 480-19-3 | Isorhamnetin | Flavonol |
| 31 | 548-83-4 | Galangin | Flavonol |
| 40 | 528-58-5 | Cyanidin | Anthocyanin |
| 45 | 38183-03-8 | 7,8-Dihydroxyflavone | Flavone |
| 46 | 151698-64-5 | 3,7,3′-Trihydroxyflavone | Flavonol |
| 57 | 501-97-3 | 3-(4- | Microbial Metabolite of |
| | | Hydroxyphenyl)propionic | proanthocyanidins |
| | | acid | |
| 58 | 86639-52-3 | SN-38 | Anticance drug metabolite |
| 61 | 38183-04-9 | 6,7-Dihydroxyflavone | Flavone |
| 66 | 520-33-2 | Hesperetin | Flavonone |
| 68 | 6068-78-6 | 3,3′,4′-Trihydroxyflavone | Flavonol |
| 70 | 1214-47-7 | 2′-hydroxychalcone | Chalcone |
| 73 | 40316-76-5 | 8-Hydroxy-7- | Flavone |
| | | methoxyflavone | |
| 77 | Not known | 4′-Hydroxy-3′- | Flavone |
| | | methoxyflavone | |
| 96 | 501-36-0 | Resveratrol | Polyphenol |
| 104 | Not known | 7-Hydroxy-6- | Isoflavone |
| | | methoxyisoflavone | |
| 157 | 622-62-8 | 4-Ethoxyphenol | Phenol |
| 119 | 29219-03-2 | 3-Hydroxy-2′- | Flavonol |
| | | methoxyflavone | |
| 121 | 632-85-9 | Wogonin | Flavone |
| 125 | 93-35-6 | 7-hydroxycoumarin | Coumarin |
| 133 | 480-19-3 | Isorhamnetin | Flavonol |
| 135 | 80-05-7 | Bisphenol A | Environmental Toxin |
| 141 | 106-41-2 | 4-bromophenol | Phenol |
| 144 | 17238-05-0 | Dihydrodaidzein | Isoflavone |
| 147 | 552-59-0 | Prunetin | Isoflavone |
| 148 | 100-02-7 | Nitrophenol | Phenol |
| 150 | 480-11-5 | Oroxylin A | Flavone |
| 157 | 622-62-8 | 4-Ethoxyphenol | Phenol |

For purposes of the disclosure herein, the term “comprising” includes “consisting” or “consisting essentially of.” Further, for purposes of the disclosure herein, the term “including” includes “comprising,” “consisting,” or “consisting essentially of.”

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

While embodiments of the invention have been shown and described, modifications thereof can be made by one skilled in the art without departing from the spirit and teachings of the invention. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, RL, and an upper limit, RU, is disclosed, any number falling within the range is specifically disclosed. In particular, the following numbers within the range are specifically disclosed: R=RL+k* (RU-RL), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined above is also specifically disclosed. Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

We claim:

1. A method of rapidly determining a UGT-transferase substrate comprising:
contacting a UGT-transferase with a UDP-sugar conjugate and a probe to form an assay mixture;
subjecting the assay mixture to reaction conditions to form a reacted mixture;
contacting the reacted mixture with a UDP-reactive dye under conditions suitable to associate the UDP with the UDP-reactive dye; and
spectrophotometrically determining an amount of free UDP, wherein if a concentration of free UDP is greater than a concentration of free UDP in a reference sample prepared in the absence of the UGT-transferase, then the probe is a designated a substrate.

2. The method of claim 1, wherein the UDP-sugar conjugate comprises UDP-glucose, UDP-glucuronic acid and UDP-rhamnose UDP-xylose, UDP-glucuronic acid, UDP-arabinose, UDP-rhamnose, UDP-galactose, GDP-fructose, GDP-mannose, or combinations thereof.

3. The method of claim 1, wherein the UDP-sugar conjugate comprises UDP-glucose.

4. The method of claim 1, wherein the conditions to form a reacted mixture comprise a pH of from about 6.5 to about 7.5.

5. The method of claim 1, wherein the conditions to form a reacted mixture comprise an amount of UGT-transferase of from about 1 μg to about 10 μg.

6. The method of claim 1, wherein the conditions to form a reacted mixture comprise an amount of UDP-sugar conjugate of from about 1 nmol to about 10 nmol.

7. The method of claim 1, wherein the sample comprises the substrate and a co-substrate, wherein the co-substrate is UDP-glucose, and wherein the method further comprises:
contacting the co-substrate comprising the UDP-glucose with the substrate without a presence of the UGT as a second sample; and
detecting a second fluorescence intensity from the second sample;
wherein detecting enzymatic activity of the UGT is based on a ratio of the fluorescence intensity to the second fluorescence intensity.

8. The method of claim 1, wherein the UDP-reactive dye is fluorescent.

9. The method of claim 1, wherein the UDP-reactive dye comprises N,N-dimethylaminoethylamino)-1,3-dioxo-1H-benzo[de]isoquinolin-2(3H)-yl)ethyl methacrylate (NI1); 2-(6-(2-(N,N-dimethylaminoethoxy)-1,3-dioxo-1H-benzo[de]isoquinolin-2(3H)-yl)ethyl methacrylate; perylene-based probe; perylene; perylene diimides (PDIs); benzo[ghi]perylene; 3,4,9,10-perylenetetracarboxylic dianhydride (PTCDA); substituted perylene derivatives; or combinations thereof.

10. The method of claim 1 wherein the UDP-reactive dye is present in an amount of form about 1 nmol to about 10 nmol.

11. The method of claim 1, wherein the probe comprises a molecule with suitable acceptor functional groups for glucuronidation.

12. The method of claim 1, wherein spectrophotometrically determining the amount of free UDP comprises preparing a calibration curve using known concentrations of free UDP.

13. A method of rapidly determining a UGT-transferase substrate comprising:
contacting a UGT-transferase with a UDP-glucose conjugate and a probe to form an assay mixture;
subjecting the assay mixture to reaction conditions to form a reacted mixture;
contacting the reacted mixture with a UDP-reactive dye under conditions suitable to associate the UDP with the UDP-reactive dye wherein the UDP-reactive dye is fluorescent; and
spectrophotometrically determining an amount of free UDP.

14. The method of claim 13, wherein the conditions to form a reacted mixture comprise a pH of from about 6.5 to about 7.5.

15. The method of claim 13, wherein the conditions to form a reacted mixture comprise a temperature of from about 30° C. to about 40° C.

16. The method of claim 13, wherein the conditions to form a reacted mixture comprise an amount of UGT-transferase of from about 1 μg to about 10 μg.

17. The method of claim 13, wherein the conditions to form a reacted mixture comprise an amount of UDP-glucose conjugate of from about 1 nmol to about 10 nmol.

18. The method of claim 13, wherein the UDP-reactive dye comprises N,N-dimethylaminoethylamino)-1,3-dioxo-1H-benzo[de]isoquinolin-2(3H)-yl)ethyl methacrylate (NI1); 2-(6-(2-(N,N-dimethylaminoethoxy)-1,3-dioxo-1H-benzo[de]isoquinolin-2(3H)-yl)ethyl methacrylate; perylene-based probe; perylene; perylene diimides (PDIs); benzo[ghi]perylene; 3,4,9,10-perylenetetracarboxylic dianhydride (PTCDA); substituted perylene derivatives; or combinations thereof.

19. The method of claim 13, wherein the probe comprises a molecule with suitable acceptor functional groups for glucuronidation.

20. The method of claim 13, further comprising:
determining that the probe is a substrate when a concentration of the amount of free UDP is greater than a concentration of free UDP in a reference sample prepared in an absence of the UGT-transferase.
